(19) United States
(12) Patent Application Publication
(10) Pub. No.: US 2022/0330249 A1
(43) Pub. Date: Oct. 13, 2022

(54) MONITORING DEVICE, WIRELESS COMMUNICATION DEVICE, AND CONTROL METHOD

(71) Applicant: SHENZHEN MINDRAY BIO-MEDICAL ELECTRONICS CO., LTD., Shenzhen (CN)

(72) Inventors: Changgen CHEN, Shenzhen (CN); Xiangjia LI, Shenzhen (CN); Heng WANG, Shenzhen (CN); Wenbin WU, Shenzhen (CN); Bin LIU, Shenzhen (CN)

(73) Assignee: SHENZHEN MINDRAY BIO-MEDICAL ELECTRONICS CO., LTD., Shenzhen (CN)

(21) Appl. No.: 17/854,047

(22) Filed: Jun. 30, 2022

Related U.S. Application Data
(63) Continuation of application No. PCT/CN2019/130959, filed on Dec. 31, 2019.

(51) Int. Cl. H04W 72/04

(52) U.S. Cl. CPC H04W 72/0453; H04W 84/18

(57) ABSTRACT

This disclosure provides a monitoring device, a wireless communication device, and a control method. The monitoring device includes a control system assembly, a digital-analog mixing assembly, a frequency band selection assembly, and an antenna. The control system assembly is respectively connected with the digital-analog mixing assembly and the frequency band selection assembly; one end of the frequency band selection assembly is connected with the digital-analog mixing assembly through a channel of designated frequency band, and the other end of the frequency band selection assembly is connected with the antenna. The monitoring device can switch from a currently used frequency band to other frequency bands for signal transmission or use other frequency bands for simultaneous signal transmission, according to a signal transmission status of the currently used frequency band. Thus, the anti-interference ability of the monitoring device can be improved to reduce the error rate of signal transmission.

### CROSS REFERENCE TO RELATED APPLICATIONS

This application is a continuation of Patent Cooperation Treaty Application No. PCT/CN2019/130959, filed on Dec. 31, 2019, which is hereby incorporated by reference in its entirety.

### TECHNICAL FIELD

The disclosure relates to the technical field of medical devices, and more particularly to a monitoring device, a wireless communication device, and a control method thereof.

### BACKGROUND

In the normal bidirectional monitoring system, the background device and the monitoring device both support bidirectional monitoring. That is, the background device and monitoring device can transmit and receive related information. For example, the background device can transmit control information to the monitoring device wirelessly, and the monitoring device can also transmit at least one of acquired target object information, device information and user information to the background device to realize the monitoring of the target object and the management of the device and user.

Although the bidirectional monitoring system does not need to deploy antennas in each room to reduce engineering installation, however in the actual monitoring process, the normal bidirectional monitoring system still has some shortcomings. For example, the normal bidirectional monitoring system has poor ability of fading depth resistance, poor anti-interference ability and high requirement for installation condition, etc. Moreover, the data transmission between the background device and the monitoring device is prone to interference, which may affect the monitoring of the target object and related first-aid operations.

### SUMMARY

In this regard, the present disclosure is aimed at the technical problem of how to improve the stability of a monitoring device and a monitoring system.

For solving its technical problem, a monitoring device is provided in this disclosure, which including a control system assembly, a digital-analog mixing assembly, a frequency band selection assembly, and an antenna;

wherein the control system assembly is respectively connected with the digital-analog mixing assembly and the frequency band selection assembly; one end of the frequency band selection assembly is connected with the digital-analog mixing assembly through a channel of designated frequency band, and the other end of the frequency band selection assembly is connected with the antenna;

the control system assembly is configured to acquire at least one of target object information, device information and user information, and to control the digital-analog mixing assembly to transmit a signal through the channel of designated frequency band, wherein the designated frequency band includes several frequency bands and the signal carries at least one of the target object information, the device information and the user information; wherein the control system assembly is further configured to control the frequency band selection assembly to turn on a channel which corresponds to the designated frequency band to transmit the signal of the designated frequency band by the antenna.

In one embodiment, the control system assembly is configured to control the digital-analog mixing assembly to generate a signal of a first frequency band and a signal of a second frequency band; wherein the control system assembly is further configured to control the frequency band selection assembly and the antenna to transmit the signal of the first frequency band and/or the signal of the second frequency band.

In one embodiment, the control system assembly includes an information processing unit and a control system unit;

wherein the information processing unit is connected with an information acquisition apparatus and is configured to acquire at least one of the target object information, the device information and the user information;

the control system unit is configured to control the digital-analog mixing assembly to generate the signal of the designated frequency band by using the target object information as signal content.

In one embodiment, the frequency band selection assembly includes a combiner and/or a switch.

In one embodiment, the switch includes a first switch and a second switch, wherein the first switch is configured to control on/off states of a first channel, and the second switch is configured to control on/off states of a second channel; the first channel and the second channel are respectively connected with the digital-analog mixing assembly and are respectively configured to transmit a signal of a first frequency band and/or a signal of a second frequency band.

In one embodiment, the digital-analog mixing assembly includes a digital-analog mixer and a radio frequency conversion circuit;

the digital-analog mixer is configured to generate a signal of a first frequency band, the radio frequency conversion circuit is configured to convert the signal of the first frequency band into a signal of another frequency band and transmit the signal of another frequency band through the frequency band selection assembly which is connected with the radio frequency conversion circuit.

In one embodiment, the digital-analog mixing assembly includes at least one radio frequency conversion circuit, which is configured to at least convert the signal of the first frequency band into a signal of a second frequency band.

In one embodiment, the digital-analog mixing assembly includes a plurality of radio frequency conversion circuits, which are respectively connected between the digital-analog mixer and the frequency band selection assembly, and are configured to convert the signal of the first frequency band into signals of a plurality of other frequency bands.

In one embodiment, the digital-analog mixer and the radio frequency conversion circuit are independent of each other; or the radio frequency conversion circuit is integrated inside the digital-analog mixer.

In one embodiment, the digital-analog mixing assembly includes a plurality of digital-analog mixers, which are connected in parallel between the control system assembly and the frequency band selection assembly, wherein the plurality of digital-analog mixers are configured to generate signals of a plurality of frequency bands respectively, and a frequency band of each signal is different.

In one embodiment, the digital-analog mixing assembly includes: a first digital-analog mixer and a second digital-analog mixer; wherein the first digital-analog mixer and the second digital-analog mixer are respectively connected between the control system assembly and the frequency band selection assembly; the first digital-analog mixer is configured to generate a signal of a first frequency band, and the second digital-analog mixer is configured to generate a signal of a second frequency band.

In one embodiment, the monitoring device is also configured to receive a communication signal which is from a monitoring system and for a wireless communication.

The present disclosure has further provided a wireless communication device, which includes at least one of a wireless access point and a monitoring center;

wherein the wireless access point and/or the monitoring center include a control system assembly, a digital-analog mixing assembly, a frequency band selection assembly, and an antenna;

wherein the control system assembly is respectively connected with the digital-analog mixing assembly and the frequency band selection assembly; one end of the frequency band selection assembly is connected with the digital-analog mixing assembly through a channel of designated frequency band, and the other end of the frequency band selection assembly is connected with the antenna;

the control system assembly is configured to acquire at least one of target object information, device information and user information, and to control the digital-analog mixing assembly to transmit a signal through the channel of designated frequency band, wherein the designated frequency band includes several frequency bands and the signal carries at least one of the target object information, the device information and the user information; wherein the control system assembly is further configured to control the frequency band selection assembly to turn on a channel which corresponds to the designated frequency band to transmit the signal of the designated frequency band by the antenna.

The present disclosure has further provided a control method of a wireless communication device, which including:

acquiring, by a control system assembly, at least one of target object information, device information and user information;

controlling, by the control system assembly, a digital-analog mixing assembly to transmit a signal through a channel of designated frequency band, wherein the designated frequency band includes several frequency bands, and the signal carries at least one of the target object information, device information and user information; and

controlling, by the control system assembly, a frequency band selection assembly to turn on a channel which corresponds to the designated frequency band to transmit the signal of the designated frequency band by an antenna.

By improving the monitoring device and monitoring system, the monitoring device and monitoring system can simultaneously transmit and receive signals of a plurality of frequency bands, such that the timeliness of signal transmission between the monitoring device and monitoring system can be improved. While improving the anti-interference ability of monitoring device and monitoring system, the construction of the monitoring system is also convenient. In such a way, the installation difficulty can also be reduced.

### BRIEF DESCRIPTION OF THE DRAWINGS

FIG. 1 is a frame diagram of a monitoring device provided in an embodiment of this disclosure.

FIG. 2 is a frame diagram of another monitoring device provided in an embodiment of this disclosure.

FIG. 3 is a frame diagram of a further monitoring device provided in an embodiment of this disclosure.

FIG. 4 is a frame diagram of another further monitoring system provided in an embodiment of this disclosure.

FIG. 5 is a frame diagram of a monitoring system and monitoring device provided by the embodiment of this disclosure.

FIG. 6 is a frame diagram of a wireless access point provided in an embodiment of this disclosure.

FIG. 7 is a frame diagram of another wireless access point provided in an embodiment of this disclosure.

FIG. 8 is a frame diagram of a further wireless access point provided in an embodiment of this disclosure.

FIG. 9 is a frame diagram of another further wireless access point provided in an embodiment of this disclosure.

FIG. 10 is a diagram of a control method provided in an embodiment of this disclosure.

### DETAILED DESCRIPTION

In order to have a clearer understanding of the technical features, purpose and effect of this disclosure, the specific embodiment of this disclosure is described in detail with reference to the accompanying drawings.

The normal monitoring device transmits signals; such as electrocardiograph (ECG) parameter, Oxygen saturation (SpO2) parameter, non-invasive blood pressure (NIBP) parameter, which are acquired from a target object; to the monitoring system through a fixed frequency band. Then the monitoring system analyzes a physical state of the target object according to these parameters. In hospitals and other places, based on considerations of etiology, physical quality and condition and so on, different target objects need different types of monitored parameters. Accordingly, the frequencies which are operable to acquire the parameters are also different. Therefore, different target objects may be provided with the monitoring devices of different types or manufacturers. Accordingly, when these monitoring devices are used, the way of transmitting signals of predetermined inherent frequency bands is easy to make the signals of the inherent frequency band to interfere with each other. This would easily lead to the distortion of the signals received by the normal monitoring system, or the failure of the normal monitoring system to receive the signal transmitted by the monitoring device. In some cases, the sudden situation of the target object, which is easy to occur, may not be known by the medical staff in time, and this may even delay the time for the medical staff to rescue the target object.

Based on the above problems, the embodiments of this disclosure provide a monitoring device and a monitoring system. The monitoring device can provide at least two frequency bands, and the medical staff can select corresponding frequency bands and transmit signals through these frequency bands. Thus, it can be guaranteed that at least one of the target object information, device information and user information acquired by the monitoring device from the target object can be timely and accurately transmitted to the corresponding monitoring system for background personnel to invoke, view, analyze or perform other operations on. If the frequency band currently used for the signal transmission is easy to be interfered or the transmission is not ideal, based on the monitoring device provided in this disclosure, the medical staff can select the other frequency band to transmit the signal, or the medical staff can select a plurality of frequency bands to transmit the signal simultaneously, so as to improve the timeliness of the signal transmission.

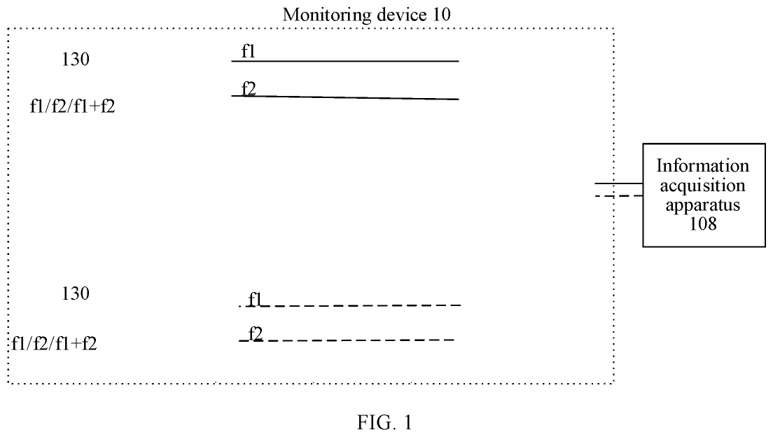

Referring FIG. 1, the monitoring device 10 which is provided in an embodiment of this disclosure, includes a control system assembly 100, a digital-analog mixing assembly 110, a frequency band selection assembly 120, and an antenna 130. The control system assembly 100 is respectively connected with the digital-analog mixing assembly 110 and the frequency band selection assembly 120. One end of the frequency band selection assembly 120 is connected with the digital-analog mixing assembly 110 through a channel of designated frequency band, and the other end of the frequency band selection assembly 120 is connected with the antenna 130. In such a way, the wireless communication between the monitoring device 10 and the monitoring system 20 is realized.

The control system assembly 100 can provide necessary electric energy and related control instructions for assemblies, such as the digital-analog mixing assembly 110, the frequency band selection assembly 120 and other assemblies in the monitoring device 10. The control system assembly 100 can acquire at least one of target object information, device information and user information, and control the digital-analog mixing assembly 110 to generate a signal of a designated frequency band, wherein the designated frequency band includes several frequency bands. The signals of these designated frequency bands all carry at least one of the target object information, the device information and the user information and include signals of one frequency band or signals of two or more frequency bands.

Take the generation of signals of a first frequency band, a second frequency band and a third frequency band by the digital-analog mixing assembly 110 under the control of the control system assembly 100 for example. The signal of the second frequency band and the signal of the third frequency band can be the signals which are generated by a radio frequency conversion circuit based on the first frequency band. Accordingly, the frequency band selection assembly 120 turns on a channel of a frequency band which corresponds to the designated frequency band to transmit the signal of the designated frequency band through the antenna 130, so as to realize the function that the monitoring device can selectively transmit the signal of the designated frequency band. This selectivity can be a frequency band selection function which is preset in the monitoring device 10 by programming at the time of delivery and then be selected, switched or connected by the medical staff. Or this selectivity can be realized through changing a usage frequency band of the monitoring device 10 by technicians when performing a related maintenance on the monitoring device 10 after delivery. In additional, the monitoring device 10 can also automatically detect a currently accessible frequency band which such as has a high signal quality/strength or a low packet loss rate, when the signal is disconnected, no data packet is received, or the signal quality/strength is lower than a preset value, so as to realize the automatic frequency band selection function. For example, the monitoring device 10 transmits the signal of the first frequency band. Alternatively, the monitoring device 10 transmits the signal of the second frequency band or the signal of the third frequency band. Alternatively, the monitoring device 10 transmits the signal of the first frequency band and the signal of the second frequency band at the same time. The present disclosure is not limited. It should be understood that the operation of the transmission includes transmitting and receiving signals of corresponding frequency bands. When the monitoring device 10 transmits signals of corresponding frequency bands, the monitoring system 20 can selectively receive signals of one frequency band, or simultaneously receive signals of these frequency bands, based on the same design concept, which will be further described below.

Compared with the normal monitoring device, the monitoring device 10 provided in this embodiment can switch from a currently used frequency band to other frequency bands for signal transmission or use other frequency bands for simultaneous signal transmission, according to a signal transmission status of the currently used frequency band. Thus, the anti-interference ability of the monitoring device 10 can be improved to reduce the error rate of signal transmission. Accordingly, the medical staff of the monitoring system 20 can also timely and accurately receive at least one of the target object information, device information and user information. In some cases, according to the signals obtained in time, medical staff can rescue the target object in the shortest time without delaying.

In order to simplify analyzing and help understanding the technical scheme of this disclosure, the monitoring device 10 and the monitoring system 20 in each embodiment are mostly exemplified to simultaneously transmit and receive signals of two frequency bands, at most. On this basis, the monitoring device 10 and the monitoring system 20 include following three operation situations which include: transmitting and receiving signals of the first frequency band f1, transmitting and receiving signals of the second frequency band f2, and transmitting and receiving signals of the first frequency band f1 and the second frequency band f2 simultaneously. However, it should also be understood that, based on the technical concept of this disclosure and exhaustive method, the monitoring device 10 and the monitoring system 20 in each embodiment can also receive and transmit signals of three or more frequency bands simultaneously.

Referring to FIG. 1, the control system assembly 100 of each monitoring device 10 is connected with two groups of digital-analog mixing assembly 110, frequency band selection assembly 120 and antenna 130. The two antennas 130 are located at different positions of the monitoring device 10, so signals are transmitted and received through the antennas 130 in different positions, so as to guarantee the normal communication between the monitoring device 10 and the monitoring system 20.

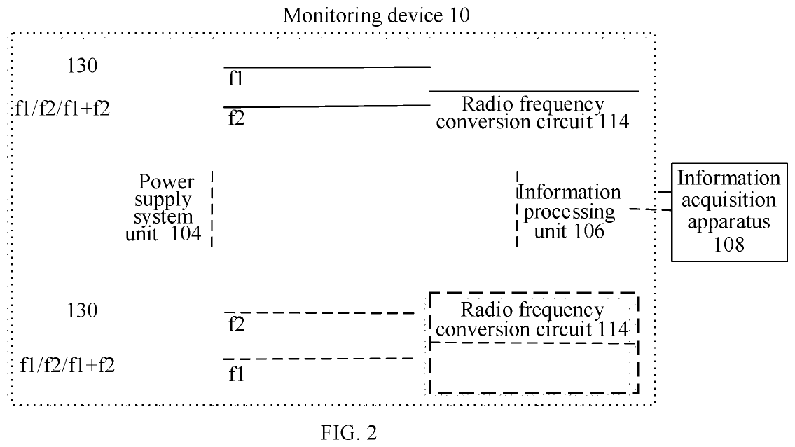

Referring to FIG. 2, in one embodiment, the control system assembly 100 includes a control system unit 102, a power supply system unit 104 and an information processing unit 106. The information processing unit 106 is in connection with an information acquisition apparatus 108 and the likes for acquiring at least one of the target object information, the device information and the user information. It should be understood that “connection” includes direct connection and indirect connection, and “acquiring” includes direct acquiring (such as acquiring by itself) and indirect acquiring (such as acquiring and then processing by itself). The information acquisition apparatus 108 can directly measure information from the target object, or acquire at least one of the target object information, user information and device information by scanning, photographing, etc., or acquire at least one of the target object information, user information and device information through an input device. After acquiring the above information, the information acquisition apparatus 108 can also process it to obtain corresponding derivative information. The acquired information may be generated in other monitoring devices and transmitted to the monitoring device 10 in the present embodiment through the information acquisition apparatus 108. For example, parameter information of the target object, such as the ECG parameters, blood oxygen saturation parameters and so on, can be acquired through a multi-parameter monitor, and then transmitted to the monitoring device 10 of this embodiment, so as to facilitate the sorting and analysis of these parameter information, or to further obtain alarm information or diagnostic information. Of course, the monitoring device 10 in this embodiment can refer to a multi-parameter monitor, and the related body information of the target object can be converted into parameter information directly through the information acquisition apparatus 108 and stored in the multi-parameter monitor.

The control system unit 102 can be configured to control the digital-analog mixing assembly 110 to generate a signal of a designated frequency band by using at least of the target object information, the device information and the user information as signal content, so as to guarantee that the signal of at least one frequency band can be accurately transmitted to the corresponding monitoring system 20 for the medical staff to invoke, review or analyze. The target object information is the information which is derived from the target object and its derivative information. The target object information includes but is not limited to physiological parameter information of the target object, motion parameter information of the target object, state parameter information of the target object, identity parameter information of the target object, alarm information which is generated due to the above parameter information of the target object and its derivative information, which is not specifically limited herein. User information includes operation information of a user, identity information of a user, authentication information of a user, request information of a user, control instruction information of a user and other information derived from the user and its derivative information. Device information refers to information representing a current state of the device. Device information includes but is not limited to such as device power information, location information, fault information and other information which is related to the device and its derivative information. In some embodiments, the control system unit 102 may input a first instruction to control the digital-analog mixing assembly 110 to generate signals of the first frequency band f1 and signals of the second frequency band f2, and the control system unit 102 may also input a second instruction to wirelessly transmit signals of the first frequency band f1 and signals of the second frequency band f2 simultaneously through the frequency band selection assembly 120 and the antenna 130. Accordingly, the antenna 130 of the monitoring system 20 can simultaneously receive the signals of the first frequency band f1 and the signals of the second frequency band f2. When the signal of the first frequency band f1 cannot be received by the monitoring system 20 due to the instability of the first frequency band f1, the monitoring system 20 still can synchronously obtain at least one of the target object information, device information and user information through the received signals of the second frequency band f2. Thus, the effectiveness of monitoring on the target object can be guaranteed.

The power supply system unit 104 is provided with a battery (not shown) and a power distribution circuit (not shown). The power distribution circuit can convert a power supply voltage provided by the battery into working voltages which are required by each functional unit in the monitoring device, and distribute the working voltages to each functional unit in the monitoring device to supply power. For example, the voltage of 15V is converted to 1.8V, 3.3V or 3.8V, so that the functional units in the monitoring device 10 can operate normally.

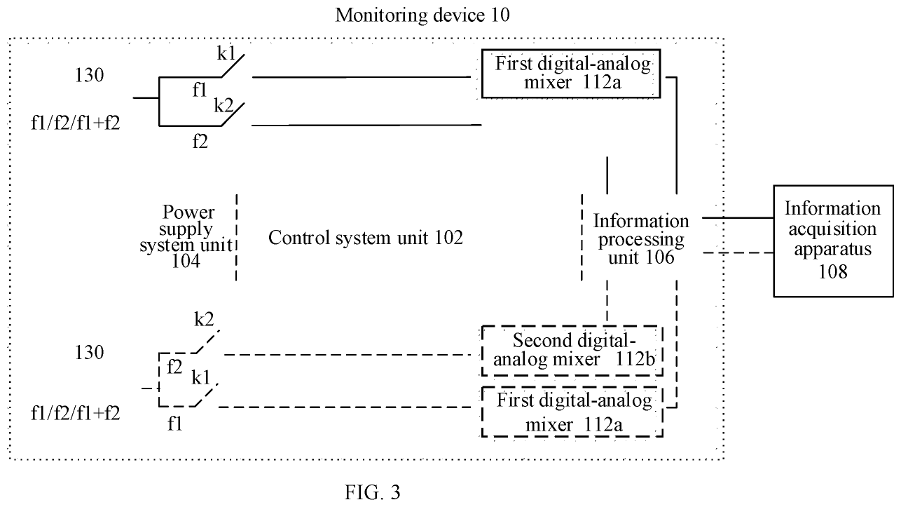

Referring to FIGS. 2 and 3, in some embodiments, the frequency band selection assembly 120 includes a switch (k1, k2) and/or a combiner 122. In FIG. 2, the frequency band selection assembly 120 includes a combiner 122, one end of the combiner 122 is provided with channels corresponding to each frequency band, and the other end of the combiner 122 is connected with the antenna 130 to transmit signals through the antenna 130. It should be understood that, based on the state of the combiner 122, the combiner 122 can transmit signals of any one certain frequency band, for example, only the signals of the first frequency band f1, or simultaneously transmit signals of a plurality of frequency bands, such as both the signals of the first frequency band f1 and the signals of the second frequency band f2.

It should be understood that the number of channels of the combiner 122 is consistent with the number of the frequency bands supported by the monitoring device 10. In some embodiments, the monitoring device 10 provided in this embodiment can support more frequency bands. According to the number of frequency bands, the combiner 122 has a corresponding number of channels, such as channels corresponding to the first frequency band f1, the second frequency band f2, the third frequency band f3, the fourth frequency band f4, etc., to support the normal operation of these frequency bands.

For example, the monitoring device 10 supports two frequency bands. Correspondingly, the combiner 122 is a dual frequency combiner and has two channels to support the transmission and receipt of signals of the two frequency bands. In some possible embodiments, assuming that the first frequency band f1 is the frequency band commonly used by other devices, the medical staff can configure the monitoring device 10 to use the second frequency band f2 or use both of the first frequency band f1 and the second frequency band f2 for communication, and the monitoring system 20 can receive the signals of the first frequency band f1 form the monitoring device 10 or other devices, as well as the signals of the second frequency band f2 from the monitoring device 10 itself to guarantee the normal operation of each device.

Of course, in some other embodiments, the combiner 122 can also be a triple frequency combiner which can transmit and receive signals of three frequency bands, which is not limited.

Referring FIG. 2, in some other embodiments, the digital-analog mixing assembly 110 includes a digital-analog mixer 112 and a radio frequency conversion circuit 114. The digital-analog mixer 112 may include a digital-analog mixing module, a digital-analog mixing chip, a digital-analog mixing circuit and any other structure, element, device or assemblies that can realize the digital-analog mixing function. In one embodiment, the digital-analog mixer 112 has a plurality of output ports, one of which is directly connected with the channel of the frequency band selection assembly 120, and the other output ports are respectively connected with the corresponding radio frequency conversion circuit 114. The radio frequency conversion circuit 114 is then connected with the frequency band selection assembly 120 to convert the signal of the first frequency band into the signal of other frequency bands. It should be understood that the digital-analog mixer 112 can generate signals of the first frequency band f1 under the control of the control system assembly 100 and transmit it to the frequency band selection assembly 120 through the channel of the frequency band selection assembly 120, then the frequency band selection assembly 120 wirelessly transmits the signal through the antenna 130. The radio frequency conversion circuit 114 is connected with other channels of the frequency band mixing assembly 120, so that the radio frequency conversion circuit 114 can convert the signals of the first frequency band f1 into signals of other frequency bands. Similarly, the signals of other frequency bands are transmitted through the corresponding channel of the frequency band selection assembly 120 and then further wirelessly transmitted through the frequency band mixing assembly 120 and the antenna 130. Based on the frequency band selection assembly 120, the monitoring device 10 of each embodiment can transmit the signal of the corresponding frequency band according to the operation of the medical staff, so as to reduce the possibility of interference of other devices on the signal, thus guaranteeing that the monitoring system 20 can receive and obtain at least one of the target object information, device information and user information in time.

In some embodiments, in order to guarantee the timeliness of signal transmission, the medical staff can control the signals of a plurality of frequency bands to be transmitted simultaneously, so as to reduce the possibility that the signal of a certain frequency band cannot be transmitted to the monitoring system 20 due to sudden interference.

In some embodiments, in order to reduce the problem that when the frequency band used by the monitoring device 10 is shared with other devices, poor signal transmission effect is resulted, the digital-analog mixing assembly 110 includes a plurality of radio frequency conversion circuits 114, which are operable to convert the signals of the first frequency band f1 into the signals of other frequency bands. It should be understood that the frequency bands which are converted by the plurality of radio frequency conversion circuits 114 are different to provide more frequency bands. In case that interference presents in the currently used frequency band, the medical staff can select other frequency bands to transmit signals.

In some embodiments, the radio frequency conversion circuit 114 can be connected with each channel, and the control system unit 102 controls the radio frequency conversion circuit 114 to configure the frequency band of each channel to the designated frequency band for information transmission. For example, the first frequency band f1 and the second frequency band f2 are respectively provided with the radio frequency conversion circuit 114, and the control system unit 102 can control the radio frequency conversion circuit 114 to switch the frequency bands of the first frequency band f1 and the second frequency band f2 to the currently available third frequency band f3, so as to realize the information transmission of other frequency bands.

In some other embodiments, different from switching to other frequency bands by using the radio frequency conversion circuit 114, the digital-analog mixing assembly 110 is employed. The digital-analog mixing assembly 110 includes a plurality of digital-analog mixers 112, which are connected in parallel between the control system assembly 100 and the frequency band selection assembly 120 to respectively process at least one of the target object information, the device information and the user information. The plurality of digital-analog mixers 112 generate signals of a plurality of frequency bands, and the frequency bands of each signal are different. Thus, under the control of the control system assembly 100, the signals of the plurality of frequency bands are selectively transmitted through the frequency band selection assembly 120 and the antenna 130. It should be understood that the plurality of digital-analog mixers 112 can also enable the monitoring device 10 to transmit the signals of the plurality of frequency bands simultaneously, so as to improve the anti-interference ability of the monitoring device 10.

Please refer to FIG. 3 and take the monitoring device 10 which supports two frequency bands for example. The digital-analog mixing assembly 110 includes: a first digital-analog mixer 112a and a second digital-analog mixer 112b. The first digital-analog mixer 112a and the second digital-analog mixer 112b are respectively connected between the control system assembly 100 and the frequency band selection assembly 120. The first digital-analog mixer 112a and the second digital-analog mixer 112b work respectively. The first digital-analog mixer 112a is operable to generate signals of the first frequency band f1, and the second digital-analog mixer 112b is operable to generate signals of the second frequency band f2. Thus, through the cooperation of the first digital-analog mixer 112a and the second digital-analog mixer 112b, the monitoring device 10 can selectively use the first frequency band f1 or the second frequency band f2. Alternatively, the monitoring device 10 can use both the first frequency band f1 and the second frequency band f2. In some possible embodiments, assuming that the first frequency band f1 is the frequency band commonly used by other devices, the medical staff can configure the monitoring device 10 to use the second frequency band f2 or use both the first frequency band f1 and the second frequency band f2 for communication, and the monitoring system 20 can receive the signal of the first frequency band f1 from the monitoring device 10 or other devices, or the signal of the second frequency band f2 from the monitoring device 10, so as to guarantee the normal operation of each device.

In addition, in some embodiments, the frequency band selection assembly 120 includes a switch. It can be understood that the switch includes a single one switch or a switch group composed of single switches, such as a single-pole double-throw (SPDT) switch, a single-pole multiple-throw (SPMT) switch, a switch group, etc. As shown in FIG. 3, the switch in this embodiment is a switch (k1, k2), one end of the switch (k1, k2) is provided with a channel which corresponds to each frequency band. That is, the channel of each frequency band is provided with switch (k1, k2) to selectively turn on or off the signal of the corresponding frequency band through the switch (k1, k2). The other end of the switch (k1, k2) is connected with the antenna 130 to transmit the signal through the antenna 130. It should be understood that, based on the state of the switch (k1, k2), the antenna 130 can only transmit signals of a certain frequency band, for example, only signals of the first frequency band f1, or alternatively the antenna 130 can simultaneously transmit signals of the plurality of frequency bands, for example, signals of the first frequency band f1 and signals of the second frequency band f2. Specifically, the switch (k1, k2) include a first switch k1 and a second switch k2, wherein the first switch k1 is operable to control on/off states of the first channel, and the second switch k2 is operable to control on/off states of the second channel. Thus, by controlling the first switch k1 and the second switch k2, the signals of the first frequency band f1 or the signals of the second frequency band f2 can be selectively transmitted, or alternatively, the signals of the first frequency band f1 and the signals of the second frequency band f2 can be transmitted simultaneously.

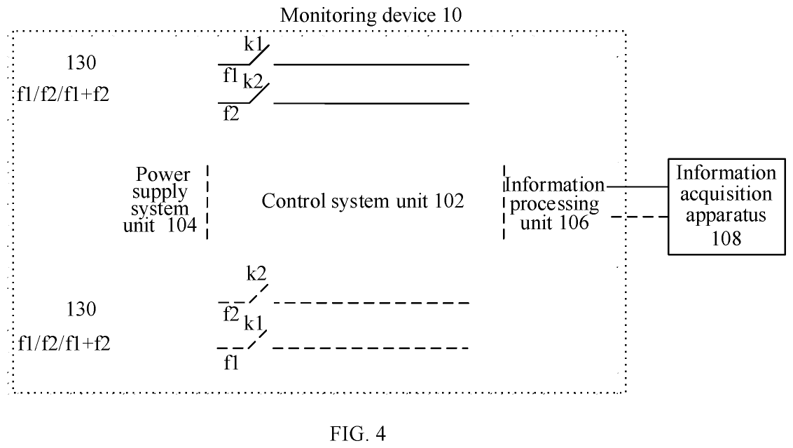

Referring to FIG. 4, in some embodiments, the radio frequency conversion circuit in the above embodiments can also be integrated inside the digital-analog mixer 112c. That is, the digital-analog mixer 112c in this embodiment is the digital-analog mixing assembly. It should be understood that the digital-analog mixer 112c of this embodiment has the same function as the combination of relatively independent digital-analog mixer 112 and radio frequency conversion circuit 114 in other embodiments. However, since the radio frequency conversion circuit is integrated into the digital-analog mixer 112c, the volume and weight of the monitoring device 10 which employs the digital-analog mixer 112c can be reduced to a certain extent. Especially when the monitoring device 10 is a wearable monitoring device, the medical staff or the target object can carry the monitoring device 10. In such a way, the portability of the monitoring device 10 can be improved.

In some embodiments, as shown in FIG. 4, the frequency band selection assembly 120 may include a combiner 122 and a switch (k1, k2). One end of the combiner 122 has channels which are corresponding to each frequency band, respectively, and each channel is provided with a switch (k1, k2) to selectively turn on or off a signal of a corresponding frequency band through the switch (k1, k2). The other end of the combiner 122 is connected with the antenna 130 to transmit the signal through the antenna 130. It should be understood that based on the state of the switch (k1, k2), the combiner 122 can transmit only signals of a certain frequency band, for example, only signals of the first frequency band f1, or alternatively, the combiner 222 can simultaneously transmit signals of the plurality of frequency bands, for example, the combiner 122 can transmit signals of the first frequency and signals of the second frequency band f2, simultaneously. Specifically, the switch (k1, k2) include a first switch k1 and a second switch k2, the first switch k1 is operable to control on/off states of the first channel, and the second switch k2 is operable to control on/off states of the second channel. Thus, by controlling the first switch k1 and the second switch k2, the signals of the first frequency band f1 or the signals of the second frequency band f2 can be selectively transmitted, or alternatively, the signals of the first frequency band f1 and the signals of the second frequency band f2 can be transmitted simultaneously.

In some embodiments, the digital-analog mixing assembly 110 of the monitoring device 10 has the function of generating signals of corresponding frequency bands, but it should also be understood that the digital-analog mixing assembly 110 can also demodulate signals of these frequency bands to receive related signals. For example, the monitoring device 10 can receive communication signals from the monitoring system.

In some embodiments, the monitoring device 10 in each embodiment may also include other elements for realizing specific functions. For example, the monitoring device 10 may include a lead wire and an electrode piece to acquire ECG parameters of the target object. The monitoring device 10 may include a data interface for wired data transmission with other monitoring devices 10 or computers. The monitoring device 10 may include a display which is operable to dynamically display at least one of the acquired target object information, device information and user information, and display a communication status with the monitoring system 20.

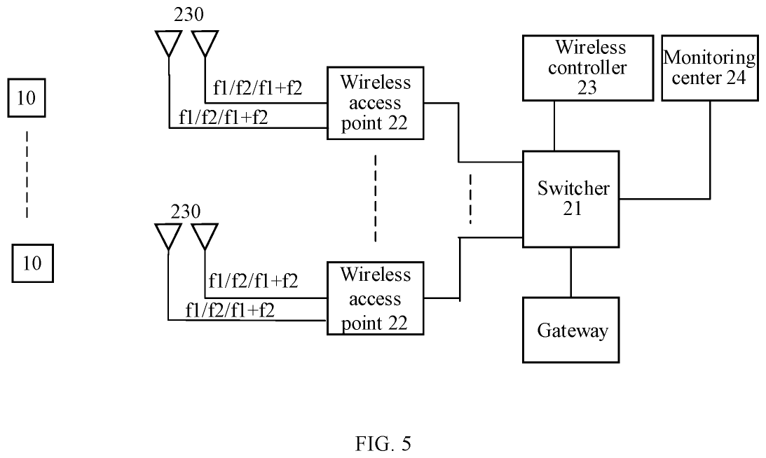

Referring to FIG. 5, the embodiment of this disclosure also provides a monitoring system 20, which can receive and transmit signals of different frequency bands to communicate with the monitoring device 10, such as receiving signals transmitted by the monitoring device 10. In addition, the monitoring system 20 may also transmit signals, which may include a control instruction for the monitoring device 10. Based on the monitoring system 20, the medical staff can realize the remote monitoring of the target object, and since the signals are modulated based on different frequency bands, it can correspondingly guarantee that the monitoring system 20 timely receives these signals and at least one of the target object information, device information and user information which are carried in these signals. In some cases, the monitoring system 20 provided by the embodiments can avoid delaying the rescue opportunity of the target object.

In some embodiments, the monitoring system 20 includes a switcher 21, a wireless access point 22, a wireless controller 23, and a monitoring center 24.

The switcher 21 is connected with the wireless access point 22, the wireless controller 23 and the monitoring center 24 respectively to guarantee the normal operation of these assemblies. The wireless access point 22 may receive a signal transmitted by the monitoring device 10 to acquire at least one of the target object information, device information and user information in the signal. For example, the wireless access point 22 can receive signals transmitted by the monitoring device 10 of the above embodiments. Accordingly, these signals can be formed based on different frequency bands. Alternatively, the wireless access point 22 may receive signals transmitted by normal monitoring device. Accordingly, these signals may be formed based on a fixed frequency band. For facilitating understanding, the monitoring system 20 in each embodiment is mostly illustrated as being in wireless communication with the monitoring device 10 in the above embodiments.

Corresponding to the monitoring device 10 in the above embodiments, in order to realize the receipt and transmission of the signals of different frequency bands, the wireless access point 22 includes a control system assembly 200, a digital-analog mixing assembly 210, a frequency band selection assembly 220 and an antenna 230. The control system assembly 200 is respectively connected with the switcher 21, the digital-analog mixing assembly 210 and the frequency band selection assembly 220. One end of the frequency band selection assembly 220 is connected with the digital-analog mixing assembly 210 through a channel, and the other end of the frequency band selection assembly 220 is connected with the antenna 230.

The antenna 230 is operable to receive signals of one frequency band or signals of two or more frequency bands simultaneously. Based on the frequency band selected by the monitoring device 10, the frequency band selection assembly 220 turns on the corresponding channel to transmit these signals, and further transmits these signals to the digital-analog mixing assembly 210. Thus, the control system assembly 200 can demodulate the signals through the digital-analog mixing assembly 210 to acquire at least one of the target object information, device information and user information in the signal. In some embodiments, the control system assembly 200 may transmit the demodulated signal through the switcher 21. For example, the demodulated signal is transmitted to the monitoring center 24 through the switcher 21.

The wireless controller 23 is operable to control the wireless access point 22 to allocate the IDs of the wireless access point 22 and the monitoring device 10, and to control the access and disconnection between the monitoring device 10 and the monitoring system 20. It should be understood that each wireless access point 22 has a certain coverage. For example, in places such as hospitals, it is often necessary to set up a plurality of wireless access points 22 to achieve a wider coverage, so as to guarantee that the signals of the monitoring device 10 can be transmitted to the monitoring system 20 in time. Thus, the wireless controller 23 can comprehensively regulate the plurality of wireless access points 22 to reduce the difficulty of constructing the monitoring system 20.

In some embodiments, the switcher 21 and the wireless controller 23 may not be used. The monitoring system of these embodiments includes a wireless communication device, wherein the wireless communication device includes at least one of the wireless access point 22 and the monitoring center 24. The access point and/or the monitoring center 24 include a control system assembly 200, a digital-analog mixing assembly 210, a frequency band selection assembly 220, and an antenna 230. The control system assembly 200 is respectively connected with the digital-analog mixing assembly 210 and the frequency band selection assembly 220. One end of the frequency band selection assembly 220 is connected with the digital-analog mixing assembly 210 through a channel of designated frequency band, and the other end of the frequency band selection assembly 220 is connected with the antenna 230. The control system assembly 200 is configured to acquire at least one of target object information, device information and user information, and to control the digital-analog mixing assembly 210 to transmit a signal through the channel of designated frequency band, wherein the designated frequency band includes several frequency bands and the signal carries at least one of the target object information, the device information and the user information. The control system assembly 200 is further configured to control the frequency band selection assembly 220 to turn on a channel which corresponds to the designated frequency band to transmit the signal of the designated frequency band by the antenna 230. In this embodiment, both of the wireless access point 22 and the monitoring center 24 can have a frequency band selection function to connect the monitoring device 10 or other wireless devices. For example, when the monitoring center 24 is a monitoring device, such as a bedside monitor, the switcher 21 and the wireless controller 23 may not be used.

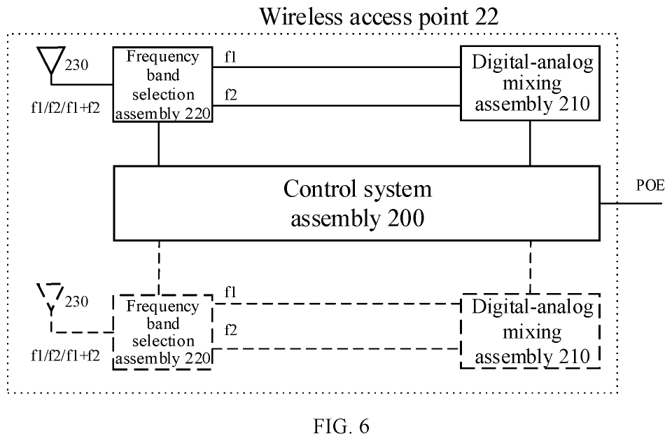

Referring to FIG. 5 and FIG. 6 simultaneously, the control system assembly 200 of the monitoring system 20 connects two groups of digital-analog mixing assemblies 210, frequency band selection assembly 220 and antenna 230. The two antennas 230 can be arranged in different corners of a hospital or other places, so as to transmit and receive signals through the antennas 230 which are spatially arranged in different positions, so as to guarantee the normal communication between the monitoring system 20 and each monitoring device 10.

The monitoring center 24 can receive the demodulated signals transmitted through the switcher 21, and also can directly receive the signals transmitted by the monitoring device 10 or the wireless device. These signals can be presented on the display for the medical staff to view timely. In some embodiments, the monitoring center 24 refers to a central station or monitoring device 10. For example, the medical staff can simultaneously check same or different information (that is, the at least one of the target object information, device information and user information) acquired by a plurality of other monitoring devices 10 on one monitoring device 10, so as to monitor the target object flexibly.

In some embodiments, for example, in places, such as hospitals, because there are many target objects, and different target objects may be matched with monitoring devices 10 of different types or manufacturers, and these many monitoring devices 10 will transmit signals frequently with multiple wireless access points 22 located in different corners. When the monitoring device 10 and the monitoring system 20 use a certain fixed frequency band, the plurality of signals of the fixed frequency band are easy to interfere with each other, thus degrading the signals and preventing the monitoring system 20 form receiving at least one of the target object information, device information and user information in time. Based on this, the wireless access point 22 and/or the monitoring center 24 provided in the embodiment of the present disclosure are similar to the monitoring device 10 in the above embodiments and can also support signals of plurality of frequency bands. The wireless access point 22 and/or the monitoring center 24 include a control system assembly 200, a digital-analog mixing assembly 210, a frequency band selection assembly 220 and an antenna 230. The control system assembly 200 is connected with the digital-analog mixing assembly 210 and the frequency band selection assembly 220 respectively. One end of the frequency band selection assembly 220 is connected with the digital-analog mixing assembly 210 through a channel, and the other end is connected with the antenna 230.

The antenna 230 can receive signals of one frequency band or signals of two or more frequency bands simultaneously. These signals may be from the monitoring device 10 which is connected with the monitoring system 20 and include at least one of target object information, device information and user information. When the scheme of the wireless access point 22 is adopted, the wireless access point 22 can receive the information of the monitoring device 10. Specifically, based on the frequency band of these signals, the frequency band selection assembly 220 turns on the corresponding channel to transmit these signals. Therefore, these signals can be demodulated by the digital-analog mixing assembly 210 for the medical staff to invoke or can be transmitted to the monitoring center 24.

Due to the limitation of its structure, the normal monitoring system can only work in a certain frequency band simultaneously. When the frequency band is interfered, for example, when there are many connected monitoring devices 10 or there are interference sources, it is easy to block the communication between the monitoring device 10 and the monitoring system 20, resulting in that the related signals of the monitoring device 10 are not transmitted to the monitoring system 20 in time. In this regard, the monitoring system 20 of each embodiment of this disclosure improves the structure of the wireless access point 22 and/or the monitoring center 24, so that the monitoring system 20 can carry out the signal transmission in a plurality of different frequency bands simultaneously, for example, the monitoring system 20 and the monitoring device 10 can realize the signal transmission on the first frequency band f1 and the second frequency band f2 simultaneously. Based on this, the monitoring system 20 provided in this disclosure can have stronger anti-interference ability. When these monitoring systems are applied to hospitals and other places, they can guarantee the effective monitoring of the target objects in the hospital, thus guaranteeing that the medical staff can rescue the target objects in time in case of accidents.

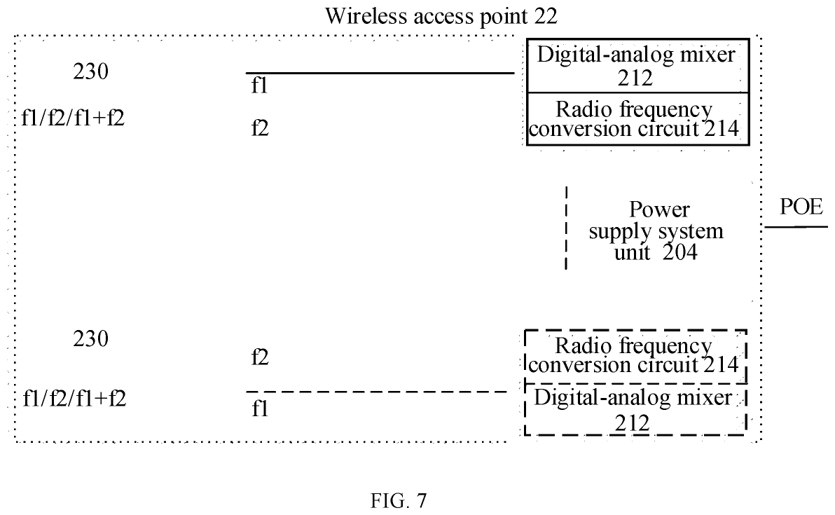

Referring to FIG. 7, in some embodiments, the control system assembly 200 of the wireless access point 22 and/or the monitoring center 24 includes a control system unit 202 and a power management unit 204.

The control system unit 202 can control the digital-analog mixing assembly 210 to demodulate the signals of designated frequency bands, wherein the signals include at least one of target object information, device information and user information, so as to timely acquire the parameter information about the target object in the corresponding signal for medical staff to review or invoke. In some embodiments, the antenna 230 of the monitoring system 20 can receive the signals of the first frequency band f1 and the signals of the second frequency band f2 simultaneously. When the signal of the frequency band f1 cannot be received by the monitoring system 20 due to the instability of the first frequency band f1, the monitoring system 20 can synchronously obtain at least one of the target object information, device information and user information through the received signal of the second frequency band f2. Thus, the effectiveness of monitoring the target object can be guaranteed.

The wireless access point 22 of each embodiment of this disclosure can realize the signal transmission and power supply through the switcher 21. Accordingly, the switcher 21 generally refers to power over ethernet (POE) switcher. Of course, the switcher 21 can also refer to other switchers that can realize the power supply. The power management unit 204 is provided with a power distribution circuit (not shown). The power distribution circuit can distribute the electric energy provided by the switcher 21 and convert it into the working voltage required by each functional unit of the wireless access point 22, so that each functional unit in the monitoring device 10 can operate normally.

In some other embodiments, the wireless access point 22 and/or the monitoring center 24 are provided with a battery (not shown), which can cooperate with the power management unit 204 to power each functional unit of the wireless access point.

Referring to FIG. 7, in some embodiments, the frequency band selection assembly 220 of the wireless access point 22 and/or the monitoring center 24 includes a switch (t1, t2) and/or a combiner 222. In FIG. 7, the frequency band selection assembly 120 includes a combiner 222. One end of the combiner 222 is provided with channels corresponding to each frequency band, and the other end of the combiner 222 is connected with the antenna 230 to transmit signals through the antenna 230. It should be understood that, based on the state of combiner 222, the combiner 222 can transmit signals of any one certain frequency band, for example, only the signals of the first frequency band f1, or simultaneously transmit signals of a plurality of frequency bands, such as both the signals of the first frequency band f1 and the signals of the second frequency band f2.

It should be understood that the number of channels of combiner 122 is consistent with the number of the frequency bands which are supported by the monitoring device 10. Alternatively, the number of channels of the combiner 222 of the monitoring system is more than the number of frequency bands supported by the monitoring device 10. In some embodiments, after the monitoring device 10 is updated or the frequency band width of the monitoring device 10 is changed by programming, accordingly, technicians or medical personnel can adaptively adjust the wireless access point 22 of the monitoring system 20 to receive the signals transmitted by the monitoring devices 10.

In some embodiments, similar to the monitoring device 10 in the above embodiments, the monitoring system 20 provided in this disclosure can support more frequency bands. According to the number of frequency bands, the combiner 222 has a corresponding number of channels, such as channels corresponding to the first frequency band f1, the second frequency band f2, the third frequency band f3, the fourth frequency band f4, etc., to support the normal operation of these frequency bands.

Similarly, the monitoring system 20 supports two frequency bands. Correspondingly, the combiner 222 is a dual frequency combiner and has two channels to support the transmission and receipt of signals of the two frequency bands. In some possible embodiments, assuming that the first frequency band f1 is the frequency band commonly used by other devices, the medical staff can configure the monitoring device 10 and the monitoring system 20 to use both of the first frequency band f1 and the second frequency band f2 for communication, and the monitoring system 20 can receive the signal of the first frequency band f1 form the monitoring device 10 or other devices, as well as the signal of the second frequency band f2 from the monitoring device 10 to guarantee the normal operation of each device.

Of course, in some other embodiments, the combiner 222 can also be a triple frequency combiner which can transmit and receive signals of three frequency bands, which is not limited.

Referring FIG. 7, in some other embodiments, the digital-analog mixing assembly 210 includes a digital-analog mixer 212 and a radio frequency conversion circuit 214. In one embodiment, after receiving the signals of the first frequency band f1 by the antenna 230, the frequency band selection assembly 220 transmits these signals to the digital-analog mixer 212. Then the digital-analog mixer 212 demodulates the signals of the first frequency band f1. When receiving the signals of the second frequency band f2, the frequency band selection assembly 220 can transmit these signals of the second frequency band f2 to the radio frequency conversion circuit 214 through another channel. The radio frequency conversion circuit 214 cooperates with the digital-analog mixer 212 to demodulate the signals of the second frequency band f2 to acquire at least one of the target information, the device information and the user information, which is similar as that in the signals of the first frequency band f1. Based on the digital-analog mixing assembly 210, the monitoring system 20 can receive the signals of the corresponding frequency band simultaneously, so as to reduce the possibility of interference of other devices on the signal, thus guaranteeing that the monitoring system 20 can receive and obtain at least one of the target object information, device information and user information in time.

In some embodiments, in order to guarantee the timeliness of signal transmission containing at least one of the target object information, device information and user information, medical personnel can configure the corresponding monitoring device 10 and monitoring system 20 to transmit signals of a plurality of frequency bands simultaneously, so as to reduce the possibility of transmission distortion caused by sudden interference of signals in a certain frequency band.

In some embodiments, when there are many devices which are connected with the monitoring system 20 and use the same frequency band for communication, the signal transmission effect of this frequency band is easy to be poor. In this regard, the digital-analog mixing assembly 210 of the wireless access point 22 and/or the monitoring center 24 also includes a plurality of radio frequency conversion circuits. Corresponding to the monitoring device 10 of the above embodiments, the plurality of radio frequency conversion circuits 214 are operable to demodulate signals of different frequency bands. Correspondingly, the different frequency bands demodulated by the plurality of radio frequency conversion circuits 214 are different, which enables the monitoring system 20 to support more frequency bands. In case that interference presents in the currently used frequency band, the medical staff can choose other frequency bands to guarantee the normal communication between the monitoring system 20 and the devices.

In some embodiments, the radio frequency conversion circuit 214 can be connected with each channel, and the control system unit 102 controls the radio frequency conversion circuit 214 to configure the frequency band of each channel to the designated frequency bands for information transmission. For example, the first frequency band f1 and the second frequency band f2 are respectively provided with the radio frequency conversion circuit 214, and the control system unit 102 can control the radio frequency conversion circuit 214 to switch the frequency bands of the first frequency band f1 and the second frequency band f2 to the currently available third frequency band f3, so as to realize the information transmission of other frequency bands.

In some other embodiments, different from switching to other frequency bands by using the radio frequency conversion circuit 214, the digital-analog mixing assembly 210 of the wireless access point 22, includes a plurality of digital-analog mixers 212, which are connected in parallel between the control system assembly 200 and the frequency band selection assembly 220 to respectively process at least one of the target object information, the device information and the user information. The plurality of digital-analog mixers 212 process signals of a plurality of frequency bands, and the frequency bands of each signal are different. Thus, under the control of the control system assembly 200, the signals of the plurality of frequency bands are selectively transmitted through the frequency band selection assembly 220 and the antenna 230. It should be understood that the plurality of digital-analog mixers 212 can also enable the monitoring system 20 to transmit the signals of the plurality of frequency bands simultaneously, so as to improve the anti-interference ability of the monitoring system 20.

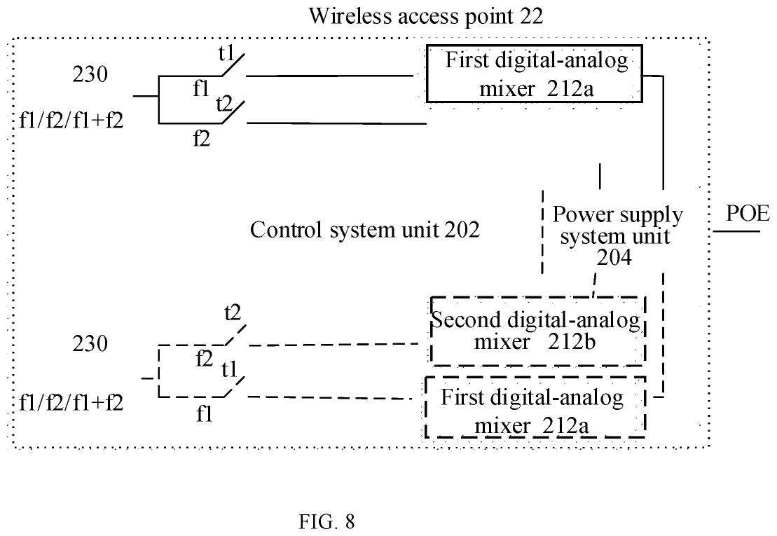

Please refer to FIG. 8 and take the monitoring system 20 which supports two frequency bands for example. The digital-analog mixing assembly 210 of the wireless access point 22 includes: a first digital-analog mixer 212a and a second digital-analog mixer 212b. The first digital-analog mixer 212a and the second digital-analog mixer 212b are respectively connected between the control system assembly 200 and the frequency band selection assembly 220. The first digital-analog mixer 212a and the second digital-analog mixer 212b work respectively. The first digital-analog mixer 212a is operable to process the signals of the first frequency band f1, and the second digital-analog mixer 212b is operable to process the signal of the second frequency band f2. Thus, through the cooperation of the first digital-analog mixer 212a and the second digital-analog mixer 212b, the monitoring system 20 can selectively use the first frequency band f1 or the second frequency band f2; or alternatively, the monitoring system 20 can use both the first frequency band f1 and the second frequency band f2. In some possible embodiments, assuming that the first frequency band f1 is the frequency band commonly used by other devices, the medical staff can configure the monitoring system 20 and the monitoring device 10 to use both the first frequency band f1 and the second frequency band f2 for communication, and the monitoring system 20 can receive the signal of the first frequency band f1 from the monitoring system 20 or other devices, or the signals of the second frequency band f2 from the monitoring device 10, so as to guarantee the normal operation of each device.

In addition, in some embodiments, the frequency band selection assembly 220 includes a switch. It can be understood that the switch includes a single one switch or a switch group composed of single switches, such as a single-pole double-throw (SPDT) switch, a single-pole multiple-throw (SPMT) switch, a switch group, etc. As shown in FIG. 8, the switch in this embodiment is a switch (t1, t2), one end of the switch (t1, t2) is provided with a channel which corresponds to each frequency band. That is, the channel of each frequency band is provided with switch (t1, t2) to selectively turn on or off the signal of the corresponding frequency band through the switch (t1, t2). The other end of the switch (t1, t2) is connected with the antenna 230 to transmit the signal through the antenna 230. It should be understood that, based on the state of the switch (t1, t2), the antenna 230 can only transmit signals of a certain frequency band, for example, only signals of the first frequency band f1, or alternatively the antenna 230 can simultaneously transmit signals of the plurality of frequency bands, for example, signals of the first frequency band f1 and signals of the second frequency band f2. Specifically, the switch (t1, t2) include a first switch t1 and a second switch t2, wherein the first switch t1 is operable to control on/off states of the first channel, and the second switch t2 is operable to control on/off states of the second channel. Thus, by controlling the first switch t1 and the second switch t2, the signals of the first frequency band f1 or the signals of the second frequency band f2 can be selectively transmitted, or alternatively, the signals of the first frequency band f1 and the signals of the second frequency band f2 are transmitted simultaneously.

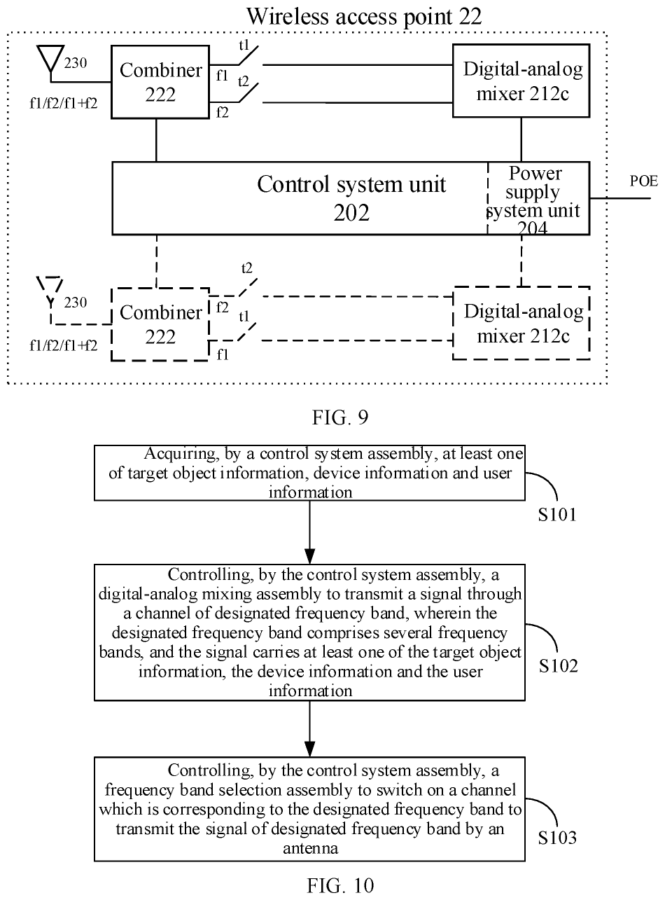

Referring to FIG. 9, in some embodiments, the radio frequency conversion circuit 214 in the above embodiments can also be integrated inside the digital-analog mixer 212c. That is, the digital-analog mixer 212c in this embodiment is the digital-analog mixing assembly. It should be understood that the digital-analog mixer 212c of this embodiment has the same function as the combination of relatively independent digital-analog mixer 212 and radio frequency conversion circuit 214 in other embodiments. However, since the radio frequency conversion circuit is integrated into the digital-analog mixer 212c, the volume and weight of the wireless access point 22 which employs the digital-analog mixer 212c can be reduced to a certain extent. In such a way, the construction, as well as the inspection and maintenance of the wireless access point 22 can be easily and implemented.

In some embodiments, as shown in FIG. 9, the frequency band selection assembly 220 may include a combiner 222 and a switch (t1, t2). One end of the combiner 222 has a channel which corresponds to each frequency band, and each channel is provided with a switch (t1, t2) to selectively turn on or off a signal of a corresponding frequency band through the switch (t1, t2). The other end of the combiner 222 is connected with the antenna 230 to transmit the signal through the antenna 230. It should be understood that, based on the state of the switch (t1, t2), the combiner 222 can transmit only signals of a certain frequency band, for example, only signals of the first frequency band f1, or alternatively, the combiner 222 can simultaneously transmit signals of the plurality of frequency bands, for example, signals of the first frequency band f1 and signals of the second frequency band f2. Specifically, the switch (t1, t2) include a first switch t1 and a second switch t2, the first switch t1 is operable to control on/off states of the first channel, and the second switch t2 is operable to control on/off states of the second channel. Thus, by controlling the first switch t1 and the second switch t2, the signals of the first frequency band f1 or the signals of the second frequency band f2 can be selectively transmitted, or alternatively, the signals of the first frequency band f1 and the signals of the second frequency band f2 can be transmitted simultaneously.

In some embodiments, the digital-analog mixing assembly 210 of the monitoring system 20 has the function of demodulating the signal of the corresponding frequency band. However, it should also be understood that the digital-analog mixing assembly 210 can also modulate the signal of this frequency band to transmit related signals. For example, the monitoring system 20 can transmit a communication signal for controlling the monitoring device 10 to implement operations such as parameter adjustment on a designated monitoring device 10.

Referring FIG. 10, the present disclosure has further provided a control method for controlling the above monitoring device, a wireless communication device, wherein the control method includes following steps:

S101. acquiring, by a control system assembly, at least one of target object information, device information and user information;

S102. controlling, by the control system assembly, a digital-analog mixing assembly to transmit a signal through a channel of designated frequency band, wherein the designated frequency band includes several frequency bands, and the signal carries at least one of the target object information, device information and user information;

S103. controlling, by the control system assembly, the frequency band selection assembly to turn on a channel which corresponds to the designated frequency band to transmit the signal of the designated frequency band by the antenna.

It can be understood that the system structure of the monitoring device, the wireless communication device and the monitoring system are shown in FIGS. 1 to 9. It can be understood that the steps S101, S102 and S103 can be in implemented in any sequence or simultaneously. There is no limitation for these herein. No matter these steps are implemented in any sequence or simultaneously, their still can satisfy the requirement that the wireless communication device can selectively transmit data in a designated frequency band, thus achieving the flexible switching and guaranteeing efficient data transmission.

In one embodiment, the method further includes: controlling, by the control system assembly, the digital-analog mixing assembly to generate a signal of a first frequency band and a signal of a second frequency band; and controlling, by the control system assembly, the frequency band selection assembly and the antenna to transmit the signal of the first frequency band and/or the signal of the second frequency band.

In one embodiment, acquiring, by a control system assembly, at least one of target object information, device information and user information, includes:

acquiring, by an information processing unit of the control system assembly, at least one of the target object information, the device information and the user information; and

controlling, by the control system assembly, the digital-analog mixing assembly to generate the signal of the designated frequency band by using the target object information as signal content.

In one embodiment, controlling, by the control system assembly, the frequency band selection assembly to turn on a channel which corresponds to the designated frequency band, includes: controlling, by the control system assembly, a combiner and/or a switch of the frequency band selection assembly to turn on the channel which corresponds to the designated frequency band.

In one embodiment, controlling, by the control system assembly, a digital-analog mixing assembly to transmit a signal through a channel of designated frequency band, includes:

controlling, by the control system assembly, a digital-analog mixer of the digital-analog mixing assembly to generate a signal of a first frequency band;

converting, by a radio frequency conversion circuit of the digital-analog mixing assembly, the signal of the first frequency band into a signal of another frequency band; and

transmitting the converted signal through the frequency band selection assembly which is connected with the radio frequency conversion circuit.

In one embodiment, controlling, by the control system assembly, a digital-analog mixing assembly to transmit a signal through a channel of designated frequency band, includes:

controlling, by the control system assembly, a plurality of digital-analog mixers of the digital-analog mixing assembly to generate signals of a plurality of frequency bands, wherein a frequency band of each signal is different.

Referring FIG. 10, the present disclosure has further provided a control method for controlling the above monitoring device, wherein the control method includes following steps:

S101. acquiring, by a control system assembly, at least one of target object information, device information and user information,

S102. controlling, by the control system assembly, a digital-analog mixing assembly to transmit a signal through a channel of designated frequency band, wherein the designated frequency band includes several frequency bands, and the signal carries at least one of the target object information, device information and user information;

S103. controlling, by the control system assembly, the frequency band selection assembly to turn on a channel which corresponds to the designated frequency band to transmit the signal of the designated frequency band by the antenna.

The above description is only the specific embodiments of the present disclosure, and the present disclosure is not limited to this. Those skilled in the art can make various changes and modifications to the present disclosure without departing from the spirit and scope of the present disclosure. Obviously, these changes and variants should fall within the protection scope required by this present disclosure. In addition, although some specific terms are used in this disclosure, however these terms are only for convenience of explanation and do not constitute any special restrictions on this disclosure

1. A monitoring device comprising a control system assembly, a digital-analog mixing assembly, a frequency band selection assembly, and an antenna;
wherein the control system assembly is respectively connected with the digital-analog mixing assembly and the frequency band selection assembly; one end of the frequency band selection assembly is connected with the digital-analog mixing assembly through a channel of designated frequency band, and the other end of the frequency band selection assembly is connected with the antenna;
the control system assembly is configured to acquire at least one of target object information, device information, or user information, and to control the digital-analog mixing assembly to transmit a signal through the channel of designated frequency band, wherein the designated frequency band includes several frequency bands and the signal carries at least one of the target object information, the device information, or the user information; wherein the control system assembly is further configured to control the frequency band selection assembly to turn on a channel, which corresponds to the designated frequency band, to transmit the signal of the designated frequency band by the antenna.

2. The monitoring device according to claim 1, wherein the control system assembly is further configured to control the digital-analog mixing assembly to generate a signal of a first frequency band and a signal of a second frequency band; wherein the control system assembly is further configured to control the frequency band selection assembly and the antenna to transmit the signal of the first frequency band or the signal of the second frequency band.

3. The monitoring device according to claim 1, wherein the control system assembly comprises an information processing unit and a control system unit;
wherein the information processing unit is connected with an information acquisition apparatus and is configured to acquire at least one of the target object information, the device information, or the user information; and
the control system unit is configured to control the digital-analog mixing assembly to generate the signal of the designated frequency band by using the target object information as signal content.

4. The monitoring device according to claim 1, wherein the frequency band selection assembly comprises a combiner or a switch;
wherein the switch comprises a first switch and a second switch, wherein the first switch is configured to control on/off states of a first channel, and the second switch is configured to control on/off states of a second channel; the first channel and the second channel are respectively connected with the digital-analog mixing assembly and are respectively configured to transmit a signal of a first frequency band or a signal of a second frequency band.

5. (canceled)

6. The monitoring device according to claim 1, wherein the digital-analog mixing assembly comprises a digital-analog mixer and a radio frequency conversion circuit;
wherein the digital-analog mixer is configured to generate a signal of a first frequency band, the radio frequency conversion circuit is configured to convert the signal of the first frequency band into a signal of another frequency band and transmit the signal of another frequency band through the frequency band selection assembly which is connected with the radio frequency conversion circuit.

7. The monitoring device according to claim 6, wherein the digital-analog mixing assembly comprises at least one radio frequency conversion circuit, which is configured to at least convert the signal of the first frequency band into a signal of a second frequency band; or
the digital-analog mixing assembly comprises a plurality of radio frequency conversion circuits, which are respectively connected between the digital-analog mixer and the frequency band selection assembly, and are configured to convert the signal of the first frequency band into signals of a plurality of other frequency bands.

8-9. (canceled)

10. The monitoring device according to claim 1, wherein the digital-analog mixing assembly comprises a plurality of digital-analog mixers, which are connected in parallel between the control system assembly and the frequency band selection assembly, wherein the plurality of digital-analog mixers are configured to generate signals of a plurality of frequency bands respectively, and a frequency band of each signal is different.

11. The monitoring device according to claim 10, wherein the digital-analog mixing assembly comprises a first digital-analog mixer and a second digital-analog mixer; wherein the first digital-analog mixer and the second digital-analog mixer are respectively connected between the control system assembly and the frequency band selection assembly; the first digital-analog mixer is configured to generate a signal of a first frequency band, and the second digital-analog mixer is configured to generate a signal of a second frequency band.

12. (canceled)

13. A wireless communication device comprising at least one of a wireless access point or a monitoring center, wherein the wireless access point or the monitoring center comprises a control system assembly, a digital-analog mixing assembly, a frequency band selection assembly, and an antenna;
wherein the control system assembly is respectively connected with the digital-analog mixing assembly and the frequency band selection assembly, one end of the frequency band selection assembly is connected with the digital-analog mixing assembly through a channel of designated frequency band, and the other end of the frequency band selection assembly is connected with the antenna;
the control system assembly is configured to acquire at least one of target object information, device information, or user information, and to control the digital-analog mixing assembly to transmit a signal through the channel of designated frequency band, wherein the designated frequency band comprises several frequency bands and the signal carries at least one of the target object information, the device information, or the user information; wherein the control system assembly is further configured to control the frequency band selection assembly to turn on a channel which corresponds to the designated frequency band to transmit the signal of the designated frequency band by the antenna.

14. (canceled)

15. The wireless communication device according to claim 13, wherein the control system assembly comprises an information processing unit and a control system unit;
wherein the information processing unit is connected with an information acquisition apparatus and is configured to acquire at least one of the target object information, the device information, or the user information; and
the control system unit is configured to control the digital-analog mixing assembly to generate the signal of the designated frequency band by using the target object information as signal content.

16. The wireless communication device according to claim 13, wherein the frequency band selection assembly comprises a combiner or a switch;
wherein the switch comprises a first switch and a second switch, wherein the first switch is configured to control on/off states of a first channel, and the second switch is configured to control on/off states of a second channel; the first channel and the second channel are respectively connected with the digital-analog mixing assembly and are respectively configured to transmit a signal of a first frequency band or a signal of a second frequency band.

17. (canceled)

18. The wireless communication device according to claim 13, wherein the digital-analog mixing assembly comprises a digital-analog mixer and a radio frequency conversion circuit;
wherein the digital-analog mixer is configured to demodulate a signal of a first frequency band, and to transmit said demodulated signal to the control system assembly; and
the radio frequency conversion circuit is configured to demodulate a signal of another frequency band in cooperation with the digital-analog mixer, and to transmit said demodulated signal to the control system assembly.

19. The wireless communication device according to claim 18, wherein the digital-analog mixing assembly comprises at least one radio frequency conversion circuit, which is configured to at least convert a signal of a second frequency band into the signal of the first frequency band; or
the digital-analog mixing assembly comprises a plurality of radio frequency conversion circuits, which are respectively connected between the digital-analog mixer and the frequency band selection assembly, and are configured to demodulate signals of a plurality of other frequency bands.

20-21. (canceled)

22. The wireless communication device according to claim 13, wherein the digital-analog mixing assembly comprises a plurality of digital-analog mixers, which are connected in parallel between the control system assembly and the frequency band selection assembly, wherein the plurality of digital-analog mixers are configured to demodulate signals of a plurality of frequency bands respectively, and a frequency band of each signal is different.

23. The wireless communication device according to claim 22, wherein the digital-analog mixing assembly comprises a first digital-analog mixer and a second digital-analog mixer; wherein the first digital-analog mixer and the second digital-analog mixer are respectively connected between the control system assembly and the frequency band selection assembly; the first digital-analog mixer is configured to demodulate a signal of a first frequency band, and the second digital-analog mixer is configured to demodulate a signal of a second frequency band.

24. (canceled)

25. A control method of a wireless communication device, comprising:
acquiring, by a control system assembly, at least one of target object information, device information, or user information;
controlling, by the control system assembly, a digital-analog mixing assembly to transmit a signal through a channel of designated frequency band, wherein the designated frequency band comprises several frequency bands, and the signal carries at least one of the target object information, the device information, or the user information; and
controlling, by the control system assembly, a frequency band selection assembly to turn on a channel which corresponds to the designated frequency band to transmit the signal of the designated frequency band by an antenna.

26. The control method according to claim 25, wherein the method further comprises:
controlling, by the control system assembly, the digital-analog mixing assembly to generate a signal of a first frequency band and a signal of a second frequency band; and
controlling, by the control system assembly, the frequency band selection assembly and the antenna to transmit the signal of the first frequency band or the signal of the second frequency band.

27. The control method according to claim 25, wherein acquiring, by a control system assembly, at least one of target object information, device information, or user information, comprises:
acquiring, by an information processing unit of the control system assembly, at least one of the target object information, the device information, or the user information; and
controlling, by the control system assembly, the digital-analog mixing assembly to generate the signal of the designated frequency band by using the target object information as signal content.

28. The control method according to claim 25, wherein controlling, by the control system assembly, a frequency band selection assembly to turn on a channel which corresponds to the designated frequency band, comprises:
controlling, by the control system assembly, a combiner or a switch of the frequency band selection assembly to turn on the channel which corresponds to the designated frequency band.

29. The control method according to claim 25, wherein, controlling, by the control system assembly, a digital-analog mixing assembly to transmit a signal through a channel of designated frequency band, comprises:
controlling, by the control system assembly, a digital-analog mixer of the digital-analog mixing assembly to generate a signal of a first frequency band,
converting, by a radio frequency conversion circuit of the digital-analog mixing assembly, the signal of the first frequency band into a signal of another frequency band, and
transmitting the converted signal through the frequency band selection assembly which is connected with the radio frequency conversion circuit; or
controlling, by the control system assembly, a digital-analog mixing assembly to transmit a signal through a channel of designated frequency band, comprises:
controlling, by the control system assembly, a plurality of digital-analog mixers of the digital-analog mixing assembly to generate signals of a plurality of frequency bands, wherein a frequency band of each signal is different.

30. (canceled)
